(19) United States
(12) Patent Application Publication
(10) Pub. No.: US 2015/0125929 A1
(43) Pub. Date: May 7, 2015

(54) USE OF CHARGED FLUOROCARBON COMPOSITIONS IN METHODS FOR PURIFICATION OF BIOMOLECULES

(71) Applicant: EMD Millipore Corporation, Billerica, MA (US)

(72) Inventors: Mikhail Kozlov, Lexington, MA (US); Wilson Moya, Concord, MA (US); Jad Jaber, Sudbury, MA (US); William Cataldo, Bradford, MA (US); Ajish Potty, Woburn, MA (US); Christopher Gillespie, Shirley, MA (US)

(21) Appl. No.: 14/362,964

(22) PCT Filed: Mar. 18, 2013
(86) PCT No.: PCT/US2013/032768

§ 371 Date: Jun. 5, 2014

Related U.S. Application Data
(60) Provisional application No. 61/615,609, filed on Mar. 26, 2012.
(60) Provisional application No. 61/666,506, filed on Jun. 29, 2012.

(51) Int. Cl. C07K 1/22; C07K 16/00; C12N 7/00

(52) U.S. Cl. CPC C07K 1/22; C12N 7/00; C07K 16/00
USPC 435/239; 530/417; 530/388.1

(57) ABSTRACT

The present invention relates to novel and improved methods for the purification of biomolecules. In particular, the present invention relates to methods of protein purification which employ a porous solid support modified with a charged fluorocarbon composition.

### RELATED APPLICATIONS

The present application claims the benefit of priority of U.S. Provisional Patent Application No. 61/615,609, filing date Mar. 26, 2012, and U.S. Provisional Patent Application No. 61/666,506, filing date Jun. 29, 2012, each of which is incorporated by reference herein in its entirety.

### FIELD

The present invention relates to novel and improved methods for purification of biomolecules. In particular, the present invention relates to methods of protein purification which employ charged fluorocarbon compositions such as, for example, sulfonated fluorinated compositions.

### BACKGROUND

The general process for the manufacture of biomolecules, such as target proteins (e.g., recombinant therapeutic proteins and antibodies), typically involves two main steps: (1) the expression of the target protein in a host cell, and (2) the purification of the target protein. The first step generally involves growing the desired host cells in a bioreactor to facilitate the expression of the target protein. Once the target protein is expressed at the desired levels, the protein is removed from the host cells and harvested. Suspended materials, such as cells, cell fragments, lipids and other insoluble matter are typically removed from the target protein-containing fluid stream by filtration or centrifugation, resulting in a clarified fluid containing the target protein in solution along with various soluble impurities. Examples of soluble impurities include host cell proteins (generally referred to as HCPs, which are cellular proteins other than the desired or targeted protein), nucleic acids, endotoxins, viruses, protein variants and protein aggregates.

The second step generally involves one or more purification steps followed by one or more polishing steps. The purification steps are generally intended to reduce the level of various soluble impurities in the clarified solution and provide concentrated target protein. The purification steps typically involve several chromatography steps, which may include one or more of bind and elute chromatography techniques, such as affinity chromatography, hydrophobic interaction chromatography (HIC), hydrophobic charge induction chromatography (HCIC), anion exchange and cation exchange chromatography, mixed mode chromatography, and can utilize resins such as ProSep-vA Ultra, ProSep Ultra Plus, MabSelect Ultra, MabSelect SuRe, SP Sepharose, Q Sepharose, Eshmuno S, Eshmuno Q, Capto Adhere, Capto MMC, HEA Hypercel, PPA Hypercel and the like.

Subsequent to subjecting a target protein-containing fluid stream to one or more purification steps, the fraction containing the target protein (referred to as the effluent) is then usually subjected to one or more polishing steps. The polishing steps are generally intended to further reduce the level of various soluble impurities in the effluent which contains the target protein. A variety of chromatography media have been reported to bind soluble impurities and are typically used in the polishing steps. For example, a simple anion-exchange chromatography media (AEX), such as one containing quarternary ammonium ligands, has been reported to bind negatively charged HCPs, DNAs, endotoxins and some viruses (see, for example, U. Gottschalk, ed., Process Scale Purification of Antibodies, John Wiley and Sons, 2009, p. 147). Chromatography resins or membranes can be used in this step, including Q Sepharose, Eshmuno Q, Fractogel TMAE, Pall Mustang Q, ChromaSorb, Sartobind Q, and the like. Further, certain “mixed mode” chromatography media have been developed which contain both anion exchange as well as cation exchange groups and may be used in the polishing steps. See, for example, F. Oehme, J. Peters, Mixed-Mode Chromatography in Downstream Process Development, BioPharm Int. Supplements, Mar. 2, 2010.

Sulfonated fluoropolymers are a unique class of macromolecules designed for applications that require unsurpassed chemical resistance and high proton conductivity. See, for example, U.S. Pat. No. 3,718,627, which describes sulfonated fluoropolymers based on the monomer CF2=CFCF2CF2SO2F. Sulfonated fluoropolymers have been previously used for surface modification of polymeric microporous membranes to improve their water wettability, to increase resistance to dewetting, and to enable their use in filtration of highly corrosive fluids, for example, as taught by U.S. Pat. No. 6,273,271.

### SUMMARY OF THE INVENTION

The present invention provides improved processes for purification of biomolecules, where the processes employ a solid support having a surface modified with a charged fluorocarbon composition, including but not limited to, fluorinated and charged polymers such as, sulfonated fluoropolymers (referred to herein as “SFPs”).

In various embodiments described herein, a porous solid support having a surface modified with a charged fluorocarbon composition is used for binding a biomolecule of interest. The biomolecule of interest which binds to a solid support modified with a charged fluorocarbon composition may either be a soluble impurity such as, for example, a host cell protein, which is intended to be removed from a target protein containing fluid stream. Alternatively, the biomolecule of interest may be a molecule that is intended to be recovered, for example, a virus or viral particle which may be used in vaccine production, where the virus or viral particle binds a solid support having a surface modified with a charged fluorocarbon composition.

In various embodiments, the present invention relates to a method of removing a biomolecule from a sample; the method comprising the steps of: (i) providing a sample comprising the biomolecule; (ii) contacting the sample with a solid support comprising a surface modified with a charged fluorocarbon composition in a flow through mode, wherein the solid support binds the biomolecule; and (iii) recovering an effluent, thereby resulting in the removal of the biomolecule.

In some embodiments, the biomolecule is an undesirable entity, e.g., a soluble impurity. In other embodiments, the biomolecule is a desirable entity, e.g., a virus or viral particle which is useful in vaccine production.

In some embodiments, the present invention relates to a method of reducing level of one or more soluble impurities in a sample, the method comprising the steps of: (i) providing a sample comprising a target protein and one or more soluble impurities; (ii) contacting the sample in a flow through mode with a porous solid support comprising a surface modified with a charged fluorocarbon composition, wherein the solid support binds the one or more soluble impurities; (iii) recovering an effluent, wherein the effluent comprises a reduced level of impurities relative to the level in (i).

In some embodiments, the level of one or more soluble impurities is reduced by at least 10%/o, or at least 20%, or at least 30%, or at least 40%, or at least 50%, or at least 60%, or at least 70%, or at least 80%, or at least 90%, or at least 95%, or more than the level present in the sample before it is contacted with a porous solid support having a surface modified with a charged fluorocarbon composition.

In yet other embodiments, the present invention relates to a method of recovering a biomolecule from a sample, the method comprising the steps of: (i) providing a sample comprising the biomolecule, (ii) contacting the sample in a flow through mode with a porous solid support comprising a surface modified with a charged fluorocarbon composition, wherein the solid support binds the biomolecule; and (iii) eluting the bound molecule from the solid support, thereby to recover the biomolecule.

In some embodiments, charged fluorocarbon composition is a sulfonated fluoropolymer.

In some embodiments, the charged fluorocarbon composition is bound to a solid support.

In some embodiments, the solid support is porous. Exemplary porous solid support formats include, but are not limited to, a membrane, a porous monolith, a woven or non-woven fabric, common chromatography resins and materials.

In various embodiments according to the present invention, the methods can be performed at a salt concentration higher than about 100 mM.

In some embodiments, following the removal of one or more soluble impurities using a porous solid support having a surface modified with a charged fluorocarbon composition, the effluent containing the target protein is further subjected to one or more chromatography or continuous chromatography steps selected from the group consisting of ion exchange chromatography, hydrophobic interaction chromatography, affinity chromatography and mixed mode chromatography.

In some embodiments, the compositions described herein are employed in a continuous process for purifying a protein from a sample (e.g., a cell culture feed). In certain embodiments, the compositions described herein are used as part of a flow-through purification process step. The flow-through purification process step may be a part of a larger protein purification process, which may include several steps including, but not limited to, e.g., culturing cells expressing protein in a bioreactor, subjecting the cell culture to clarification, which may employ one or more of precipitation, centrifugation and/or depth filtration; transferring the clarified cell culture to a bind and elute chromatography capture step (e.g., Protein A affinity chromatography); subjecting the Protein A eluate to virus inactivation (e.g., using one or more static mixers and/or surge tanks); subjecting the output from virus inactivation to a flow-through purification process, which employs two or more matrices selected from activated carbon, anion exchange chromatography media, cation exchange chromatography media and virus filtration media; and formulating the protein in the flow-through from the flow-through purification step using diafiltration/concentration and sterile filtration. Additional details of such processes can be found, e.g., in co-pending application having reference no. P12/107, filed concurrently herewith, and the entire contents of which are incorporated by reference herein. In some embodiments, the CFC compositions described herein are used before a cation exchange media during flow-through purification. In another embodiment, the CFC compositions described herein are used after a cation exchange media during flow-through purification step.

In some embodiments, a fluid sample continuously flows through the entire process, as described above, from one step to the next.

Exemplary target proteins include, but are not limited to, recombinant proteins, monoclonal antibodies and functional fragments, humanized antibodies, chimeric antibodies, polyclonal antibodies, multispecific antibodies, immunoadhesin molecules and CH2/CH3 region-containing proteins. The target proteins may be expressed in a mammalian expression system (e.g., CHO cells) or a non-mammalian expression system (e.g., bacterial, yeast or insect cells). The methods described herein may be used in the context of proteins expressed using mammalian expression systems as well as non-mammalian expression systems.

### BRIEF DESCRIPTION OF THE DRAWINGS

FIG. 1 is a schematic of a general chemical structure of a group present in the charged fluorocarbon composition. Here, R₁ is selected from a group containing sulfonic, sulfate, phosphonic, phosphoric, and carboxylic residues; R₂ is an optional linking unit that is selected from the group containing short saturated and unsaturated hydrocarbon groups, such as, e.g., CₓH_{y}, where x ranges from 1 to about 10 and y ranges from 0 to about 20, alkoxy groups, esters, amides, and the like; and R₃ is a F, Cl or a C₁ to C₁₀ perfluoroalkyl radical.

FIG. 2 represents schematic of exemplary perfluorinated monomers that can be polymerized or copolymerized to obtain a precursor of a polymeric charged fluorocarbon composition.

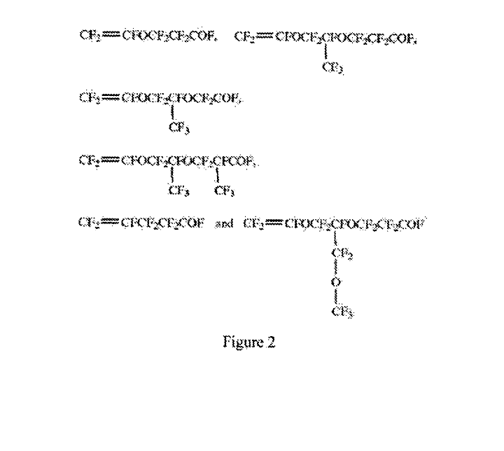

FIG. 3 represents exemplary fluorinated organic acids.

FIGS. 4A and 4B represent graphs based on the results of an exemplary experiment to measure static Lysozyme capacity of CFC-modified membranes (EW 830, EW 1000 and EW 1100) relative to unmodified membrane, as measured at pH 5 (4A) or pH 8 (4B).

FIGS. 5A and 5B represent graphs based on the results of an exemplary experiment to measure static BSA capacity of CFC-modified membranes (EW 830, EW 1000 and EW 1100) relative to unmodified membrane, as measured at pH 5 (5A) or pH 8 (5B).

FIG. 6 represents a graph based on the results of an exemplary experiment to measure static IgO capacity of CFC-modified membranes measured at pH 5 and 8 in buffer solutions containing 0.5M sodium chloride.

FIG. 7 represents a graph based on the results of an exemplary experiment to measure static Host cell protein capacity of CFC-modified membranes compared to unmodified membranes

FIGS. 8A and 8B represent graphs based on the results of an exemplary experiment to measure monoclonal antibody yield and LRV of host cell protein reduction at low conductivity (25 mM buffer, shown in 8A) or at high conductivity (25 mM buffer+250 mM NaCl, shown in 8B).

### DETAILED DESCRIPTION

The present invention is based, at least in part, on the discovery of the use of a solid support having a surface modified with a charged fluorocarbon composition (CFC) in processes for protein purification, where the processes reduce the number of polishing steps that may be used. Further, the polishing steps in the methods described herein can be performed under conditions (e.g., high salt concentration) which lead to undesirable results with other commercially available products designed for use in polishing steps. Lastly, the molecules described herein for use in protein purification processes may be less expensive to use than currently available products which may be used in a similar fashion.

Chromatography resin compositions that incorporate sulfonic acid groups, as well as sulfonic acid groups that are distinctly physically separated from perfluorinated groups have been described in the art (see, e.g., U.S. Pat. No. 8,092,683). However, such resins do not appear to exhibit any substantial binding of a target protein (i.e., insulin).

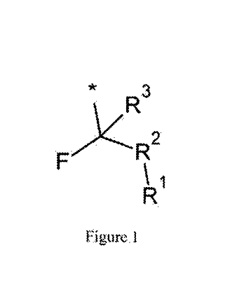

In contrast, the compositions according to the present invention include charged groups (e.g., sulfonic groups) in close proximity of fluorinated groups (e.g., fluorocarbon groups), as shown in FIG. 1. In other words, in case of the compositions described herein, the charged and the fluorinated groups are not separate and distinct groups as in case of the compositions described in the art.

In order that the present disclosure may be more readily understood, certain terms are first defined. Additional definitions are set forth throughout the detailed description.

### I. DEFINITIONS

The term “charged fluorocarbon composition,” as used herein, refers to a compound containing carbon and fluorine atoms as well as one or more charged groups, including but not limited to carboxylic, sulfonic, sulfate, phosphonic, phosphoric acid. Such fluorocarbon compositions include, in particular, saturated, unsaturated, and cyclic perfluorocarbons.

An exemplary charged fluorocarbon composition represents a class of copolymers of tetrafluoroethylene and perfluorovinyl ether, terminated with sulfonate groups. The chemical structure of these copolymers, as shown in a figure below, includes a perfluorinated polymer backbone and a strong cation exchange side group. The linker between the side group and the perfluorinated backbone varies in length and depending on the copolymer grade and the manufacturer. For example, DuPont Nafion® comonomer has the chemical structure: Perfluoro-3.6-dioxa-4-methyl-7-octene sulfonyl fluoride (MW=446): CF₂═CF—O—CF₂—CF(CF₃)OCF₂CF₂—SO₂F, while Solvay Solexis Aquivion® monomer has the chemical structure: Perfluoro-3-oxa-4-pentene sulfonyl fluoride (MW=280): CF₂═CF—O—CF₂CF₂—SO₂F.

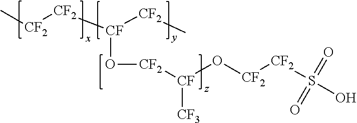

x, y, and z define the equivalent weight (grams/mole —SO₃H)
y=1, x=5.5 to 6.5
z=1 (Nafion®), 0 (Aquivion™)

These copolymers have been reported to be used in electrolysis membranes in Chlor-alkali production of chlorine and base. They have also been reported to be used in fuel cell membranes, sensors (e.g. carbon monoxide and methanol), actuators, Donnan dialysis, organic chemistry catalysis, and gas & vapor diffusion (drying/humidification of gases). Further, they have been used for surface modification of polymeric microporous membranes to improve their water wettability and increase resistance to dewetting. For example, as described in U.S. Pat. No. 6,273,271, incorporated by reference herein, they could be applied as a coating on the surface of a PTFE membrane for filtration of highly corrosive fluids.

The term “solid support,” as used herein, refers in general to a porous or a non porous material to which a charged fluorocarbon composition can adhere or be covalently bound. The solid support to which a charged fluorocarbon composition is bound or attached is referred to as a “modified solid support.” Such modified solid supports are useful for binding a biomolecule in a sample. Exemplary solid supports that may be modified include, but are not limited to, common chromatography resin materials such as, for example, agaroses, polysaccharides, dextrans, silica gels, synthetic polymers (polystyrene-divinylbenzene, polyacrylate, polymethacrylate, polyacrylamide, polyvinyl alcohol, polysulfone, polycarbonate, polyvinyl ether and their corresponding copolymers), inorganic and ceramic materials and glass beads. The solid support may be in any suitable format including, but is not limited to, a purification column, discontinuous phase of discrete particles, packed bed column, and expanded bed column. Solid supports may also include porous membranes such as microporous and ultrafiltration membranes. The ultrafiltration and microporous membranes can be in any of several forms, including sheets, tubes, and hollow fibers. Further, solid supports may include fibrous materials such as fibers or fabrics, which can be woven or non-woven.

The terms “modify,” “modified,” and “modification” as used interchangeably herein, refer to the process of changing the original surface properties of a solid support. For example, in some embodiments, the solid support may be modified by subjecting the support to priming, coating or treatment such as chemical or radiation treatment.

The term “in situ polymerization,” as used herein, refers to a process involving a polymerization reaction that is carried out on a material that has been functionalized during the same process run; I.e., the material is not removed from the reactor between the functionalizing and polymerization reactions.

The term “monomer” as used here, refers to a molecule which can join with others of the same kind to form a polymer. A monomer may join with other monomers of the same kind to form a “homopolymer.” Alternatively, a monomer may join with monomers that are not of the same kind, to form a “copolymer.” The term “monomer,” as used herein, is also intended to use starting materials of more than one monomer (referred to as “oligomers”) which are capable of joining or polymerizing with other monomers or oligomers to form a polymer. The term “dimer,” as used herein, refers to two monomers that are joined together. Similarly, the terms “trimer,” “tetramer,” and “pentamer” refer to a joinder of three, four and five monomers, respectively.

The term “grafting” as used herein refers to a polymerization reaction in which one or more species of a newly created polymer block are connected to the main chain of a macromolecule as side-chains having constitutional or configurational features that differ from those in the main chain.

As used herein, the term “crosslinking” refers to a reaction involving sites or groups on existing macromolecules or an interaction between existing macromolecules that result in the formation of a small region in a macromolecule from which at least four chains emanate. Such interactions may occur in many different ways including formation of a covalent bond, formation of hydrogen bonds, hydrophobic, hydrophilic, ionic or electrostatic interaction.

As used herein, the term “adsorption” refers to a process in which a molecule becomes attached to the surface of a solid support through physical interactions. Such interactions may occur in many different ways including formation of hydrogen bonds, hydrophobic, hydrophilic, ionic or electrostatic interaction.

The term “biomolecule” or “biomolecule of interest” or “target biomolecule,” as used interchangeably herein, is intended to be referred to any biological entity that binds to or is capable of being bound to a porous solid support having a surface modified with a charged fluorocarbon composition. In some embodiments, the biomolecule is a molecule that is desired to be removed from a sample containing a target protein such as, for example, a host cell protein. In other embodiments, the biomolecule is a molecule that is desired to be recovered from a sample such as, for example, a virus or viral particle that can be used for vaccine production.

The term “target protein,” “desired product,” “protein of interest,” or “product of interest,” as used interchangeably herein, generally refers to a polypeptide or product of interest, which is desired to be purified or separated from one or more undesirable entities, e.g., one or more soluble impurities, which may be present in a sample containing the polypeptide or product of interest. The terms “target protein,” “protein of interest,” “desired product” and “product of interest,” as used interchangeably herein, generally refer to a therapeutic protein or polypeptide, including but not limited to, an antibody that is to be purified using the methods described herein. In some embodiments, the target protein does not bind to a porous solid support having a surface modified with a charged fluorocarbon composition and, accordingly, ends up in the effluent that is recovered following the removal of one or more soluble impurities using a porous solid support having a surface modified with a charged fluorocarbon composition.

As used herein interchangeably, the term “polypeptide” or “protein,” generally refers to peptides and proteins having more than about ten amino acids. In some embodiments, a small molecule, as described herein, is used to separate a protein or polypeptide from one or more undesirable entities present in a sample along with the protein or polypeptide. In some embodiments, the one or more entities are one or more impurities which may be present in a sample along with the protein or polypeptide being purified. As discussed, above, in some embodiments according to the methods described herein, a charged fluorocarbon composition attached to a solid support is used for binding the one or more impurities (e.g., soluble impurities) in a sample comprising a target protein.

In some embodiments, a protein or polypeptide being purified using the methods described herein is a mammalian protein, e.g., a therapeutic protein or a protein which may be used in therapy. Exemplary proteins include, but are not limited to, for example, renin; a growth hormone, including human growth hormone and bovine growth hormone; growth hormone releasing factor, parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor, anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor -alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; Dnase; IgE; a cytotoxic T-lymphocyte associated antigen (CTLA), such as CTLA-4; inhibin; activin; vascular endothelial growth factor (VEGF); receptors for hormones or growth factors; Protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as α-FGF and β-FGF; epidermal growth factor (EGF); transforming growth factor (TGF) such as TGF-alpha and TGF-beta, including TGF-β1, TGF-β2, TGF-β3, TGFβ4, or TGF-β5; insulin-like growth factor-1 and -II (IGF-1 and IGF-II); des(1-3)-IGF-1 (brain IGF-1), insulin-like growth factor binding proteins (IGFBPs); CD proteins such as CD3, CD4, CD8, CD19 CD20, CD34, and CD40; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (Ils), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments and/or variants of any of the above-listed polypeptides.

Further, in some embodiments, a protein or polypeptide purified using the methods described herein is an antibody, functional fragment or variant thereof. In some embodiments, a protein of interest is a recombinant protein containing an Fc region of an immunoglobulin.

The term “immunoglobulin,” “Ig” or “IgO” or “antibody” (used interchangeably herein) refers to a protein having a basic four-polypeptide chain structure consisting of two heavy and two light chains, said chains being stabilized, for example, by interchain disulfide bonds, which has the ability to specifically bind antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable,” based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions.” The “constant” domains of antibody light chains are referred to interchangeably as “light chain constant regions,” “light chain constant domains,” “CL” regions or “CL” domains. The “constant” domains of antibody heavy chains are referred to interchangeably as “heavy chain constant region,” “heavy chain constant domains,” “CH” regions or “CH” domains. The “variable” domains of antibody light chains are referred to interchangeably as “light chain variable regions,” “light chain variable domains,” “VL” regions or “VL” domains. The “variable” domains of antibody heavy chains are referred to interchangeably as “heavy chain variable regions,” “heavy chain variable domains,” “VH” regions or “VH” domains.

Immunoglobulins or antibodies may be monoclonal (referred to as a “MAb”) or polyclonal and may exist in monomeric or polymeric form, for example, IgM antibodies which exist in pentameric form and/or IgA antibodies which exist in monomeric, dimeric or multimeric form. Immunoglobulins or antibodies may also include multispecific antibodies (e.g., bispecific antibodies), and antibody fragments so long as they retain, or are modified to comprise, a ligand-specific binding domain. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. When produced recombinantly, fragments may be expressed alone or as part of a larger protein called a fusion protein. Exemplary fragments include Fab, Fab′, F(ab′)2, Fc and/or Fv fragments. Exemplary fusion proteins include Fc fusion proteins.

Generally, an immunoglobulin or antibody is directed against an “antigen” of interest. Preferably, the antigen is a biologically important polypeptide and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) are also contemplated. Where the antigen is a polypeptide, it may be a transmembrane molecule (e.g. receptor) or a ligand such as a growth factor.

The term “monoclonal antibody” or “MAb,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). “Monoclonal antibodies” may also be isolated from phage antibody libraries using the techniques described in Clackson el al., Nature 352:624-628 (1991) and Marks et al., J. Mol. Biol. 222:581-597 (1991), for example.

Monoclonal antibodies may further include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The term “hypervariable region” when used herein refers to the amino acid residues of an antibody which are responsible for antigen-binding. The hypervariable region comprises amino acid residues from a “complementarity determining region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5^{th} Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, an antibody which is separated or purified using methods described herein, is a therapeutic antibody. Exemplary therapeutic antibodies include, for example, trastuzumab (HERCEPTIN™, Genentech, Inc., Carter et al (1992) Proc. Natl. Acad. Sci. USA, 89:4285-4289; U.S. Pat. No. 5,725,856); anti-CD20 antibodies such as chimeric anti-CD20 “C2B8” U.S. Pat. No. 5,736,137); rituximab (RITUXAN™), ocrelizumab, a chimeric or humanized variant of the 2H7 antibody (U.S. Pat. No. 5,721,108; WO 04/056312) or tositumomab (BEXXAR™); anti-IL-8 (St John et al (1993) Chest, 103:932, and WO 95/23865); anti-VEGF antibodies including humanized and/or affinity matured anti-VEGF antibodies such as the humanized anti-VEGF antibody huA4.6.1 bevacizumab (AVASTN™, Genentech, Inc., Kim et al (1992) Growth Factors 7:53-64, WO 96/30046, WO 98/45331); anti-PSCA antibodies (WO 01/40309); anti-CD40 antibodies, including S2C6 and humanized variants thereof (WO 00/75348); anti-CD 11a (U.S. Pat. No. 5,622,700; WO 98/23761; Steppe et al (1991) Transplant Intl. 4:3-7; Hourmant et al (1994) Transplantation 58:377-380); anti-IgE (Presta et al (1993) J. Immunol. 151:2623-2632; WO 95/19181); anti-CDI8 (U.S. Pat. No. 5,622,700; WO 97/26912); anti-IgE, including E25, E26 and E27 (U.S. Pat. No. 5,714,338; U.S. Pat. No. 5,091,313; WO 93/04173; U.S. Pat. No. 5,714,338); anti-Apo-2 receptor antibody (WO 98/51793); anti-TNF-alpha antibodies including cA2 (REMICADE™), CDP571 and MAK-195 (U.S. Pat. No. 5,672,347; Lorenz et al (1996) J. Immunol. 156(4):1646-1653; Dhainaut et al (1995) Crit. Care Med. 23(9):1461-1469); anti-Tissue Factor (TF) (EP 0 420 937 B1); anti-human alpha 4 beta 7 integrin (WO 98/06248); anti-EGFR, chimerized or humanized 225 antibody (WO 96/40210); anti-CD3 antibodies such as OKT3 (U.S. Pat. No. 4,515,893); anti-CD25 or anti-tac antibodies such as CHI-621 SIMULECT™ and ZENAPAX™ (U.S. Pat. No. 5,693,762); anti-CD4 antibodies such as the cM-7412 antibody (Choy et al (1996) Arthritis Rheum 39(1):52-56); anti-CD52 antibodies such as CAMPATH-1H (Riechmann et al (1988) Nature 332:323.337); anti-Fe receptor antibodies such as the M22 antibody directed against Fc gamma RI as in Graziano et al (1995) J. Immunol. 155(10):4996-5002; anti-carcinoembryonic antigen (CEA) antibodies such as hMN-14 (Sharkey et al (1995) Cancer Res. 55(23Suppl): 5935s-5945s; antibodies directed against breast epithelial cells including huBrE-3, hu-Mc 3 and CHL6 (Ceriani et al (1995) Cancer Res. 55(23):5852s-5856s; and Richman et al (1995) Cancer Res. 55(23 Supp): 5916s-5920s); antibodies that bind to colon carcinoma cells such as C242 (Litton et al (1996) Eur J. Immunol. 26(1):1-9); anti-CD38 antibodies, e.g. AT 13/5 (Ellis et al (1995) J. Immunol. 155(2):925-937); anti-CD33 antibodies such as Hu M195 (Jurcic et al (1995) Cancer Res 55(23 Suppl):5908s-5910s and CMA-676 or CDP771; anti-CD22 antibodies such as LL2 or LymphoCide (Juweid et al (1995) Cancer Res 55(23 Suppl):5899s-5907s); anti-EpCAM antibodies such as 17-IA (PANOREX™); anti-GpIIb/IIIa antibodies such as abciximab or c7E3 Fab (REOPRO™); anti-RSV antibodies such as MEDI-493 (SYNAGIS™); anti-CMV antibodies such as PROTOVIR™); anti-HIV antibodies such as PR0542; anti-hepatitis antibodies such as the anti-Hep B antibody OSTAVIR™); anti-CA 125 antibody OvaRex; anti-idiotypic GD3 epitope antibody BEC2; anti-alpha v beta3 antibody VITAXIN™; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); and anti-human leukocyte antigen (HLA) antibodies such as Smart IDIO and the anti-HLA DR antibody Oncolym (Lym-1).

The terms “contaminant,” “impurity,” and “debris,” as used interchangeably herein, generally refers to any foreign or objectionable material, including a biological macromolecule such as a DNA, an RNA, one or more host cell proteins (HCPs or CHOPs), whole cells, cell debris and cell fragments, endotoxins, viruses, lipids and one or more additives which may be present in a sample containing a protein or polypeptide of interest (e.g., an antibody) being separated from one or more of the foreign or objectionable molecules using a porous solid support having a surface modified with a charged fluorocarbon composition, as described herein.

The term “insoluble impurity,” as used herein, refers to any undesirable or objectionable entity present in a sample containing a target biomolecule, wherein the entity is a suspended particle or a solid. Exemplary insoluble impurities include whole cells, cell fragments and cell debris.

The term “soluble impurity,” as used herein, refers to any undesirable or objectionable entity present in a sample containing a target biomolecule (e.g., a target protein), wherein the entity is not an insoluble impurity. Exemplary soluble impurities include host cell proteins, DNA, RNA, viruses, endotoxins, cell culture media components, lipids etc. In some embodiments, the soluble impurity is a host cell protein (HCP).

In various embodiments described herein, a porous solid support having a surface modified with a charged fluorocarbon composition is used for binding a soluble impurity.

Without wishing to be bound by theory, it is contemplated that in certain instances, a biomolecule that binds to a solid support having a surface modified with a charged fluorocarbon composition is the biomolecule which is desired to be recovered (e.g., in case of a virus or a viral particle which is used for vaccine production).

The term “composition,” “solution” or “sample,” as used herein, generally refers to a mixture of a target protein or a product of interest to be purified along with one or more undesirable entities or impurities (e.g., soluble proteins). In some embodiments, the sample comprises a biological material containing stream, e.g., feedstock or cell culture media into which a target protein or a desired product is secreted. In some embodiments, the sample comprises a target biomolecule (e.g., a therapeutic protein or an antibody) along with one or more soluble impurities (e.g., host cell proteins). In some embodiments, the sample comprises a target biomolecule which is secreted into the cell culture media. In other embodiments, the sample comprises a target biomolecule which is a virus or a viral particle. In some embodiments, the sample contacted with or flowed through the compositions described herein is an output from a previous step in a purification process. For example, in a particular embodiment, the sample constitutes an output from a cation exchange flow-through chromatography step or an output from an anion exchange flow-through chromatography step.

The term “process step” or “unit operation,” as used interchangeably herein, refers to the use of one or more methods or devices to achieve a certain result in a purification process. Examples of process steps or unit operations which may be employed in purification processes include, but are not limited to, clarification, bind and elute chromatography, virus inactivation, flow-through purification and formulation. It is understood that each of the process steps or unit operations may employ more than one step or method or device to achieve the intended result of that process step or unit operation. In some embodiments, one or more devices which are used to perform a process step or unit operation are single-use devices and can be removed and/or replaced without having to replace any other devices in the process or even having to stop a process run.

The term “surge tank” as used herein refers to any container or vessel or bag, which is used between process steps or within a process step (e.g., when a single process step comprises more than one step); where the output from one step flows through the surge tank onto the next step. Accordingly, a surge tank is different from a pool tank, in that it is not intended to hold or collect the entire volume of output from a step; but instead enables continuous flow of output from one step to the next. In some embodiments, the volume of a surge tank used between two process steps or within a process step in a process or system described herein, is no more than 25% of the entire volume of the output from the process step. In another embodiment, the volume of a surge tank is no more than 10% of the entire volume of the output from a process step. In some other embodiments, the volume of a surge tank is less than 35%, or less than 30%, or less than 25%, or less than 20%, or less than 15%, or less than 10% of the entire volume of a cell culture in a bioreactor, which constitutes the starting material from which a target molecule is to be purified.

The term “continuous process,” as used herein, refers to a process for purifying a target molecule, which includes two or more process steps (or unit operations), such that the output from one process step flows directly into the next process step in the process, without interruption, and where two or more process steps can be performed concurrently for at least a portion of their duration. In other words, in case of a continuous process, as described herein, it is not necessary to complete a process step before the next process step is started, but a portion of the sample is always moving through the process steps. The term “continuous process” also applies to steps within a process step, in which case, during the performance of a process step including multiple steps, the sample flows continuously through the multiple steps that are necessary to perform the process step. One example of such a process step described herein is the flow through purification step which includes multiple steps that are performed in a continuous manner, e.g., flow-through activated carbon followed by flow-through AEX media followed by flow-through CEX media followed by flow-through virus filtration. The compositions described herein may be employed in such a flow-through purification step.

The term “static mixer” refers to a device for mixing two fluid materials, typically liquids. The device generally consists of mixer elements contained in a cylindrical (tube) housing. The overall system design incorporates a method for delivering two streams of fluids into the static mixer. As the streams move through the mixer, the non-moving elements continuously blend the materials. Complete mixing depends on many variables including the properties of the fluids, inner diameter of the tube, number of mixer elements and their design etc. In some embodiments, one or more static mixers are used throughout a purification process.

The terms “chinese hamster ovary cell protein” and “CHOP,” as used interchangeably herein, refer to a mixture of host cell proteins (“HCP”) derived from a Chinese hamster ovary (“CHO”) cell culture. The HCP or CHOP is generally present as a soluble impurity in a cell culture medium or lysate (e.g., a harvested cell culture fluid containing a protein or polypeptide of interest (e.g., an antibody or immunoadhesin expressed in a CHO cell). Generally, the amount of CHOP present in a mixture comprising a protein of interest provides a measure of the degree of purity for the protein of interest. Typically, the amount of CHOP in a protein mixture is expressed in parts per million relative to the amount of the protein of interest in the mixture.

It is understood that where the host cell is another mammalian cell type, an E. coli, a yeast cell, an insect cell, or a plant cell, HCP refers to the proteins, other than target protein, found in a lysate of the host cell. In general, the charged fluorocarbon compositions described herein can be used for binding any molecule which has a charge opposite to the compositions.

The term “parts per million” or “ppm,” as used interchangeably herein, refers to a measure of purity of a desired target molecule (e.g., a target protein or antibody) purified using methods described herein. Accordingly, this measure can be used either to gauge the amount of a target molecule present after the purification process or to gauge the amount of an undesired entity.

The term “log reduction value”, or “LRV”, as used interchangeably herein, refers to a measure of reduction of impurity concentration in a certain process. The impurity concentration can be expressed in weight per volume units, such as mg/ml, ng/ml, g/L etc., as well as number of particles per volume units, such as CFU/ml and PFU/ml (“colony forming units” and “plaque forming units”, respectively). In general, LRV may be defined as follows: LRV=Log[(Impurity concentration in feed)/(Impurity concentration in effluent)].

The terms “isolating,” “purifying” and “separating,” are used interchangeably herein, in the context of purifying a target biomolecule (e.g., a protein of interest) from a composition or sample comprising the target biomolecule and one or more impurities (e.g., host cell proteins), using a porous solid support having a surface modified with a sulfonated and charged molecule. In some embodiments, the degree of purity of the target biomolecule in a sample is increased by removing (completely or partially) one or more soluble impurities (e.g., host cell proteins) from the sample by using a solid support having a surface modified with a charged fluorocarbon composition, as described herein. In another embodiment, the degree of purity of the target biomolecule (e.g., a virus useful for vaccine production) in a sample is increased by binding the target biomolecule away from one or more soluble impurities in the sample using a porous solid support having a surface modified with a charged fluorocarbon composition, as described herein. The bound target biomolecule can subsequently be recovered by eluting it from the solid support using suitable conditions known in the art.

In some embodiments, a purification process additionally employs one or more “chromatography steps.” Typically, these steps may be carried out, if necessary, after the separation of a target biomolecule from one or more undesired entities using a solid support having a surface modified with a charged fluorocarbon composition, as described herein.

In some embodiments, a “purification step” to isolate, separate or purify a polypeptide or protein of interest using a porous solid support having a surface modified with a charged fluorocarbon composition, as described herein, may be part of an overall purification process resulting in a “homogeneous” or “pure” composition or sample, which term is used herein to refer to a composition or sample comprising less than 100 ppm HCP in a composition comprising the protein of interest, alternatively less than 90 ppm, less than 80 ppm, less than 70 ppm, less than 60 ppm, less than 50 ppm, less than 40 ppm, less than 30 ppm, less than 20 ppm, less than 10 ppm, less than 5 ppm, or less than 3 ppm of HCP.

The term “clarification,” or “clarification step,” as used herein, generally refers to one or more initial steps in the purification of a biomolecule. The clarification step generally comprises removal of whole cells and/or cellular debris using one or more steps including any of the following alone or various combinations thereof, e.g., centrifugation and depth filtration, precipitation, flocculation and settling. Clarification step generally involves the removal of one or more undesirable entities and is typically one or the first steps performed during a purification process. Another key aspect of clarification is the removal of insoluble components in a sample which may later on result in the fouling of a sterile filter in a purification process, thereby making the overall purification process more economical. In some embodiments, methods according to the present invention employ one or more of the conventional clarification steps commonly used, e.g., depth filtration and centrifugation, prior to employing a solid support modified with a charged fluorocarbon composition for removal of one or more biomolecules from a sample.

The term “chromatography,” as used herein, refers to any kind of technique which separates an analyte of interest (e.g., a target biomolecule) from other molecules present in a mixture. Usually, the analyte of interest is separated from other molecules as a result of differences in rates at which the individual molecules of the mixture migrate through a stationary medium under the influence of a moving phase, or in bind and elute processes.

The term “chromatography resin” or “chromatography media” are used interchangeably herein and refer to any kind of phase or material which separates an analyte of interest (e.g., a target biomolecule) from other molecules present in a mixture. Usually, the analyte of interest is separated from other molecules as a result of differences in rates at which the individual molecules of the mixture migrate through a stationary phase, which is generally of a solid state, under the influence of a moving phase, or in bind and elute processes. Examples of various types of chromatography media include, for example, cation exchange resins, affinity resins, anion exchange resins, anion exchange membranes, hydrophobic interaction resins and ion exchange monoliths.

The term “capture step” or “capture,” as used herein, generally refers to a method used for binding a target biomolecule. In some embodiments, the capture step involves employing a solid support having a surface modified with a charged fluorocarbon composition molecule to bind a target biomolecule, where the target biomolecule is the desired molecule to be recovered. In some embodiments, the capture step is performed subsequent to the removal of one or more impurities using a solid support having a surface modified with a charged fluorocarbon composition, where the target molecule is captured from the effluent subsequent to the removal of impurities. In other embodiments, the capture step is performed prior to the removal of one or more impurities using a solid support having a surface modified with a charged fluorocarbon composition, where the target molecule is further purified subsequent to the capture step.

### II. EXEMPLARY CHARGED FLUOROCARBON COMPOSITION

The methods according to the present invention employ charged fluorocarbon compositions in protein purification processes.

In some embodiments, the present invention relates to a method of separating a target biomolecule from one or more soluble impurities in a sample and employs a solid support modified with a charged fluorocarbon composition. Accordingly, a sample comprising a target biomolecule (e.g., a target protein or an antibody) and one or more soluble impurities is contacted with a porous solid support having a surface modified with a charged fluorocarbon compositions, wherein the soluble impurities bind to the solid support, whereas the target molecule is recovered in the effluent.

In some other embodiments, the present invention relates to a method of separating a target biomolecule from one or more undesirable entities, where the target biomolecule itself binds to a porous solid support having a charged fluorocarbon composition on its surface.

Non-limiting examples of charged fluorocarbon compositions include, but are not limited to, polymer compositions such as homopolymer or copolymers such as those marketed by E. I. Dupont de Nemours and Company, Inc. under the name NAFION®, by Solvay Solexis under the name Aquivion™ PFSA or by Asahi Glass Company, Limited under the name FLEMION™, fluorocarbon copolymers, such as those comprising at least two monomers with one monomer being selected from a group of fluorine-containing monomers such as vinyl fluoride, hexafluoropropylene, vinylidene fluoride, trifluoroethylene, chlorotrifluoroethylene, perfluoro(alkylvinyl ether), tetrafluoroethylene and mixtures thereof, and a second monomer, which may be selected from a group of fluorine-containing monomers containing functional groups which are or which can be converted to (SO₃ M) group wherein M is H, an alkali metal, or an alkaline earth metal. Examples of such second monomers can be generically represented by the formula CF₂═CFR_{f}—X. R_{f}, in the general formula is a linear or branched bifunctional perfluorinated radical comprising one to eight carbon atoms of any suitable or conventional configuration including those containing ether linkages and which is attached to the vinyl radical CF₂═CF group directly through a carbon-carbon bond or preferably through an ether linkage. X is a functional group which is or which can be converted to (SO₃ M), wherein M is H, an alkali metal, or an alkaline earth metal. One restraint upon the general formula is a requirement for the presence of at least one fluorine atom on the carbon atom adjacent the X group.

Typically second monomers contain sulfonyl fluoride groups which can be converted to sulfonyl based ion exchange groups, examples of which can be found in U.S. Pat. Nos. 3,282,875; 3,041,317; 3,560,568; and 3,718,627 which are incorporated herein by reference. Methods of preparation of perfluorocarbon polymers are set forth in U.S. Pat. Nos. 3,041,317; 2,393,967; 2,559,752 and 2,593,583 which are incorporated herein by reference. These perfluorocarbon copolymers generally have pendant SO₂F based functional groups which can be converted to (SO₃M) groups. In some embodiments, compositions according to the present invention include pendant carbonyl based functional groups which can be converted to carbonyl based ion exchange groups.

Fluorocarbon copolymers having pendant carbonyl based ion exchange functional groups can be prepared in any suitable conventional manner such as in accordance with U.S. Pat. Nos. 4,465,533 and 4,349,422 which are incorporated herein by reference. Representative examples of carbonyl fluoride containing monomers include the following monomeric formulae.

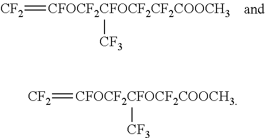

In some embodiments, fluorocarbon copolymers described herein include carbonyl and/or sulfonyl based functional groups represented by the formula —OCF₂ CF₂ X′ and/or —OCF₂CF₂C—F₂ Y—B—YCF₂ CF₂ O—, wherein X′ is sulfonyl fluoride (SO₂F), carbonyl fluoride (COF) sulfonate methyl ester (SO₃ CH₃), carboxylate methyl ester (COOCH₃), ionic carboxylate (COO—Z⁺) or ionic sulfonate (SO₃—Z⁺), Y is sulfonyl (SO₂) or carbonyl (CO), B is a linkage such as —O—, —O—O—, —S—S—, and Z is hydrogen, an alkali metal such lithium, cesium, rubidium, potassium and sodium or an alkaline earth metal such as barium, beryllium, magnesium, calcium, strontium and radium or a quaternary ammonium ion.

The sulfonyl form of the fluorocarbon copolymer is typically a polymer having a fluorinated hydrocarbon backbone chain to which are attached the functional groups or pendant side chains which, in turn, carry the functional groups. The pendant side chains can include the following structures:

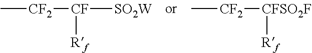

wherein R′_{f}, is F, Cl, or a C₁ to C₁₀ perfluoroalkyl radical, and W is F or Cl, preferably F. Ordinarily, the functional group in the side chains of the polymer will be present in groups which can be attached to the side chain through an ether linkage.

Examples of perfluorocarbon copolymers of this kind are disclosed in U.S. Pat. Nos. 3,282,875; 3,560,568 and 3,718,627 which are incorporated herein by reference.

Additional examples of polymers can be represented by the general formula CF₂═CF-Tₖ-CF₂SO₂F, where T is a bifunctional fluorinated radical comprising 1 to 8 carbon atoms, and k is 0 or 1. Substituent atoms in T include fluorine, chlorine, or hydrogen. In some embodiments, perfluorocarbon copolymers are free of both hydrogen and chlorine attached to carbon, i.e., they are perfluorinated, which enables the greatest stability in harsh environments. The T radical of the formula above can be either branched or unbranched, i.e., straight-chain, and have one or more ether linkages. In some embodiments, the vinyl radical in this group of sulfonyl fluoride containing comonomers is joined to the T group through an ether linkage, I.e., that the comonomer comprises the formula: CF₂═CF—O-T-CF₂—SO₂F. An exemplary sulfonyl fluoride containing comonomers can be represented by the following structures.

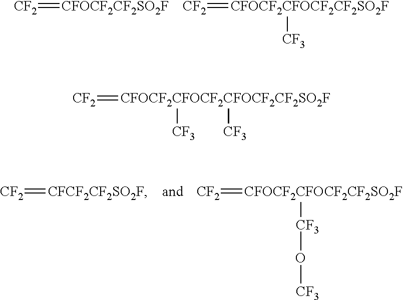

In some embodiments, a sulfonyl fluoride containing comonomer is perfluoro (3,6-dioxa-4-methyl-7-octenesulfonyl fluoride) having the following structure.

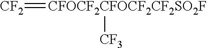

The sulfonyl-containing monomers are disclosed in such references as U.S. Pat. Nos. 3,282,875, 3,041,317 and U.S. Pat. No. 3,560,568 which are incorporated herein by reference.

In some embodiments, a class of perfluorocarbon copolymers utilized in the present invention is represented by polymers having repeating units as shown below,

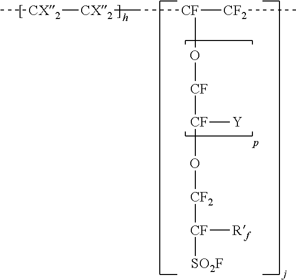

where, h is 3 to 15,j is I to 10, p is 0, 1 or 2, ‘X″’ represents four fluorines or three fluorines and one chlorine, Y is F or CF₃, and R′_{f} is F, Cl or a C₁ to C₁₀ perfluoroalkyl radical.

Any fluorocarbon polymer and copolymer which contains sulfonyl or carbonyl based functional groups can be used in the methods described herein including copolymers which contain both types of functional groups and mixtures of copolymers having different functional groups. One such example is a copolymer of tetrafluoroethylene and perfluoro (3,6-dioxa-4-methyl-7-octenesulfonyl fluoride), from which the sulfonic acid form or the salt form can be obtained. Another example is a copolymer of tetrafluoroethylene and methyl perfluoro (4,7-dioxa-5-methyl-8-nonenoate), from which the carboxylic acid form or the salt form can be obtained.

Generally, sulfonyl, carbonyl, sulfonate and carboxylate esters; and sulfonyl and carbonyl based amide forms of the perfluorocarbon copolymer are readily converted to ion exchange forms by a hydrolysis reaction. For example, the salt form can be obtained by treatment with a strong alkali such as NaOH and the acid form can then be generated by treatment with an acid such as HCl. This conversion step can be carried out before or after a solid support has been modified with the sulfonyl, carbonyl, sulfonate and carboxylate esters and sulfonyl and carbonyl based amide forms of the perfluorocarbon copolymer.

In another embodiment, the fluorocarbon compositions comprise fluorocarbon monomers described above, which are polymerized or co-polymerized in situ to coat a solid support surface.

The solvent utilized to form the reactant fluorocarbon solution from which the porous solid support surface modification is derived includes the solvents; disclosed in U.S. Pat. No. 4,386,987, which is incorporated herein by reference. These solvents include Halocarbon Oil, perfluorooctanoic oil, N-akylacetamides and decafluorobiphenyl. Alternatively, the halogenated saturated hydrocarbons disclosed in U.S. Pat. No. 4,348,310, which is incorporated herein by reference, can be utilized. In some embodiments, the solvents are the alcoholic solvents disclosed in U.S. Pat. Nos. 4,433,082 and 4,453,991, which are incorporated herein by reference. The alcoholic solvents include methanol, ethanol, n-propanol, isopropanol, n-butanol, 2-butanol, 2-methoxy ethanol, 2-ethoxy ethanol, ethylene glycol dimethyl ether, ethylene glycol diethyl ether, diethylene glycol ditnethyl ether, diethylene glycol diethyl ether, dioxane and acetonitrile and mixtures thereof with or without water. In a particular embodiment, the solvent is a mixture of water and a lower alcohol such as isopropanol. The solutions of the perfluorocarbon copolymers are formed at elevated temperature, typically 180° C. to 300° C., below the critical temperature of the solvent and at an elevated pressure in a closed vessel. These solutions are miscible with solvents or diluents for the perfluorocarbon copolymers, such as isopropanol, ethanol, water or the like, without precipitating the perfluorocarbon copolymer. It is generally required that the solution completely enter the substrate pores.

### III. EXEMPLARY SOLID SUPPORTS

Suitable media materials include those which are typically used for chromatographic beads, including glass, such as borosilicate glass, alkali resistant glass and controlled pore glass, natural and synthetic polymers such as polystyrene, polyethylene, polypropylene, blends of polyethylene and polypropylene, multilayered polyethylene/polypropylene beads, acrylics, polysulfones, polyethersulfones, PVDF or PTFE, styrene-divinyl benzene copolymer, agarose, agarose derivatives, agar, alginate, cellulose, cellulose derivatives, dextran, starch, carrageenan, guar gum, gum arabic, gum ghatti, gum tragacanth, karaya gum, locust bean gum, xanthan gum, pectins, mucins, heparins, and gelatins, metals such as stainless steel, nickel, titanium, palladium and cobalt or various iron, iron containing or other magnetized metals alloys and blends; and ceramics, such as silicate materials, zirconia and various ceramic blends. In cases where the solid support is in the form of a bead, the beads can be formed of the same material throughout, or can be a composite of two or more materials, preferably porous materials. For example, a bead can comprise a glass or synthetic polymer core on the inside and a natural and synthetic polymer layer on the outside.

In a particular embodiment, a solid support employed in the compositions and methods according to the present invention is a porous membrane. In general, the porous membrane may be comprised of any suitable material including one or more polymers. Representative suitable polymers for forming a porous membrane include polyolefins such as polyethylene, polypropylene, polymethylpentene, or the like; polystyrene or substituted polystyrenes; fluorinated polymers including poly(tetrafluoroethylene), polyvinylidene fluoride or the like; polysulfones such as polysulfone, polyethersulfone or the like; polyesters including polyethylene terephthalate, polybutylene terephthalate or the like; polyacrylates and polycarbonates; vinyl polymers such as polyvinyl chloride and polyacrylonitriles; cellulosics such as cellulose, nitrocellulose, and cellulose acetate; polyamides. Copolymers may also be used to form a bulk matrix of a porous material including copolymers of butadiene and styrene, fluorinated ethylene-propylene copolymer, ethylene-chlorotrifluoroethylene copolymer or the like.

Generally, a porous membrane employed in the methods and compositions described herein has an average pore size ranging from 0.001 to 50 microns, from 0.1 to 5 microns, or from 0.01 to 1 micron. In some embodiments the average pore size is 0.2 microns. In a particular embodiment, the average pore size of a membrane is 0.45 microns. In other embodiments, the average pore size is 0.65 microns. The membrane depth, i.e. the distance between the two outer surfaces, or top and bottom surfaces of the membrane, may range from 1 to 1000 microns, from 50 to 500 microns, from 75 to 200 microns or from 90 to 150 microns.

### IV. EXEMPLARY METHODS OF MODIFYING A SOLID SUPPORT WITH A CHARGED FLUOROCARBON COMPOSITION

In various embodiments, a solid support is modified with a charged fluorocarbon composition. A solid support can be modified using a variety of surface modification techniques known to the skilled in the art.

In some embodiments, adsorption, which is a commonly used method of surface modification, is employed. A solution of a fluorocarbon polymer composition, as described herein, is contacted with the porous solid support such as by immersion of the solid support in the solution or by passing the solution through the solid support or be intruding the pores of the solid support under pressure. By a solution herein is meant a liquid composition which contains a completely dissolved and/or partially dissolved fluorocarbon composition in a solvent, diluent or dispersant medium. These solutions include suspensions of an undissolved fluorocarbon polymer composition in a dispersant medium. The solution includes a liquid composition which is a solvent, diluent or dispersant medium for the fluorocarbon polymer composition which either completely wets the solid support or, when it does not wet the solid support, the membrane is prewet such that the solution can enter the pores or the solution is intruded into the pores. It is generally required that the solution completely enter the pores of the solid support. The solid support is contacted with the solution for a required period of time, which may range from 1 second to 24 hours, or between 1 minute and 60 minutes. In various embodiments, the time for which the solid support is contacted with the solution is 1 second or 5 seconds or 10 seconds or 20 seconds or 30 seconds or 40 seconds or 50 seconds or 1 minute or 5 minutes or 10 minutes or 15 minutes or 20 minutes or 25 minutes or 30 minutes or 35 minutes or 40 minutes or 45 minutes or 50 minutes or 55 minutes or 1 hour or 5 hours or 10 hours or 15 hours or 20 hours or 24 hours or longer. The solution is subsequently removed by mechanical means, such as nipping, squeegee, or applying a slotted die.

In an alternative embodiment, the excess of solution is not removed by mechanical means and instead is left in the pores for drying and subsequent extraction. A diluent or dispersant which selectively removes the unbound fluorocarbon polymer composition, such as by solvation or dilution, while avoiding removal of fluorocarbon composition which is bound to the solid support, is generally used.

The resultant surface modified solid support is subsequently dried and heat treated to improve the strength of binding between the membrane substrate and the bound charged fluorocarbon composition.

If the fluorocarbon polymer composition used is of the sulfonyl or carbonyl fluoride type, an additional hydrolysis step may be required to convert the surface to the ion exchange form.

The heat-treated surface modified solid support has its surface modified with a composition comprising an adsorbed fluorocarbon polymer composition which, surprisingly, is not substantially soluble in those solvents or diluents which solvate and/or dilute the unbound solvated charged perfluorocarbon polymer composition.

In addition, the surface modifying composition is utilized in amounts and concentrations such that the solid support is not substantially blocked or plugged. When the solid support is a membrane, blocking can be measured by an increase in pressure drop across the membrane during filtration of purified water.

The following procedure describes a general method for coating a solid support with a fluorocarbon composition.

A suitable solid support substrate is pre-wetted with methanol, rinsed in water, and soaked in the reactant solution comprising fluorocarbon composition for several minutes to assure complete exchange. If the reactant solution is capable of wetting the substrate directly, the prewet exchange steps are not necessary. The surface modified solid support is then subjected to removal of the excess of any unbound fluorocarbon composition, such as by mechanical compression, air knife, or any other suitable means known in the art.

In some embodiments, the excess unbound fluorocarbon composition is removed by using one or both of a mechanical force and/or treatment with a solvent, diluent or dispersant which selectively removes, such as by solvation or dilution, unbound fluorocarbon composition while avoiding removal of fluorocarbon composition which is bound to the porous support. The resultant surface modified porous support is subsequently dried and heat treated to improve the strength of binding between the substrate and the bound fluorocarbon composition.

If the fluorocarbon composition used is not inherently charged, e.g., comprising a sulfonyl halide group, an additional hydrolysis step is required to convert the surface to the ion exchange form.

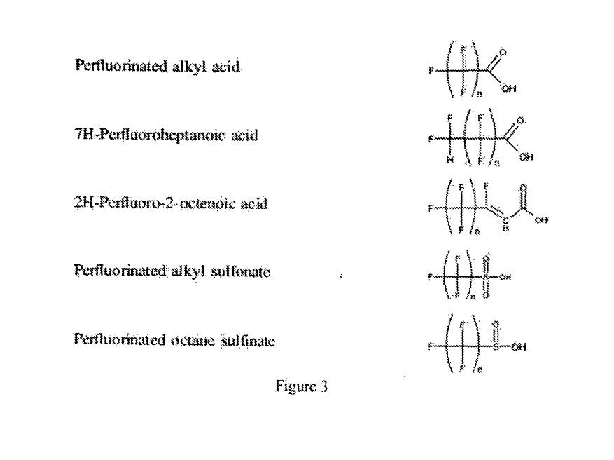

Utilizing fluorophilic interactions is yet another adsorption technique to generate modified solid supports with charged fluorocarbon compositions. For example, specific interaction of perfluorinated (PF) compounds on a stationary phase has been reported. See, e.g., De Miguel et al., Chromatographia, Vol 24, 849-853, 1987. A strong cooperative effect is observed with branched fluorinated compounds. This is especially useful when the fluorocarbon composition is a ligand that lacks polymerizable functionality. Suitable support materials include various fluorocarbon polymers, such as, polytetrafluoroethylene (PTFE) (e.g. Teflon®, registered trademark of E. I. du Pont de Nemours and Company), polyvinylfluoride and polyvinylidene difluoride and perfluorodecalin. Suitable ligand fluorocarbon compositions include, but are not limited to the compounds depicted in FIG. 3, and disclosed by Weiss J. M. et al. TOXICOLOGICAL SCIENCES 109(2), 206-216, 2009), which is incorporated herein by reference.

The following procedure describes a general method for coating a solid support with a charged fluorocarbon composition utilizing fluorophilic interactions.

A solid support substrate is wetted in methanol, rinsed in water, and soaked in the reactant solution comprising ligand fluorocarbon composition for several minutes to assure complete exchange. If the reactant solution is capable of wetting the substrate directly, the prewet exchange steps are not necessary. The surface modified solid support is then subjected to mechanical removal of the excess of unbound fluorocarbon composition.

In some embodiments, the excess unbound fluorocarbon composition is removed by using one or both of a mechanical force and/or treatment with a solvent, diluent or dispersant which selectively removes, such as by solvation or dilution, unbound fluorocarbon composition while avoiding removal of fluorocarbon composition which is bound to the porous support. The resultant surface modified porous support is subsequently dried and heat treated to improve the strength of binding between the substrate and the bound fluorocarbon composition.

If the small molecule fluorocarbon composition used is of the sulfonyl or carbonyl fluoride type, an additional hydrolysis step is required to convert the surface to the ion exchange form.

In another embodiment, surface modification of a porous solid support is carried out by crosslinking a fluorocarbon composition on the interior pore surfaces as well as the exterior, geometric surfaces.

A porous solid support can be modified with a reactant solution preferably comprising polymerizable fluorocarbon composition, and a suitable cross-linker, for example N,N′-methylenebisacrylamide (MBAm). Generally, the charged fluorocarbon composition is present in the reactant solution at a concentration between about 1 wt % and about 20 wt %, or between about 3 wt % and about 6 wt % based upon the weight of the reactant solution. The cross-linking agent is preferably present in the reactant solution in a concentration between about 5 wt % and about 100 wt % based upon the weight of the fluorocarbon composition.

The reactant solution is polymerized in situ on the surface of the porous substrate, as well as the inner pore walls, in the absence of any chemical polymerization free radical initiator, upon exposure to electron beam radiation. Preferably, the polymerized cross-linked fluorocarbon composition covers the entire outer and inner surfaces of the porous substrate. The polymerization relies upon exposing the reactant solution saturating the inner and outer surfaces of the substrate to electron beam radiation, at a dose of at least about 0.1 Mrads to about 6 Mrads, in order to effect polymerization of the fluorocarbon composition.

The following procedure describes a general method for coating a porous substrate with a fluorocarbon composition utilizing electron beam radiation. The substrate is wetted in methanol, rinsed in water, and soaked in the reactant solution comprising polymerizable fluorocarbon composition and MBAm cross-linkers for several minutes to assure complete exchange. If the reactant solution is capable of wetting the substrate directly, the prewet exchange steps are not necessary.

The electron beam technology used for initiating polymerization of the reactant solution on the surfaces of the substrate include for example, methods described in U.S. Pat. No. 4,944,879 to Steuck, the disclosure of which is incorporated herein by reference. The foregoing patent discusses, for example, a continuos roll of membrane (referred to as a web) or individual sample passed through a curtain of electrons generated by an electron beam processor. The processor delivers the desired dose from about 100 kV to about 200 kV. The moving web or sample is transported at a speed suitable to give the desired exposure time under the curtain. Exposure time, combined with dose, determines the dose rate. Typical exposure times are from about 0.5 seconds to about 10 seconds. Dose rates generally are from 0.01 kGy (kiloGray) to about 6 kGy (i.e., 0.1 to about 6 Mrads). After the desired dose of radiation has been delivered by the electron beam, the treated porous substrate is rinsed in water and/or methanol to remove unreacted and oligomeric materials. The substrate is then dried and tested for rewet, flow and other properties.

In yet another alternative embodiment, polymerization can be initiated by employing a free-radical chemical initiator instead of the ionizing radiation. When a free-radical initiator is used, it can be added to the reactant solution prior to wetting the porous substrate, typically in the amount ranging from 0.01 to 1%. Depending on the chemical nature of the free-radical initiator, the polymerization reaction can be initiated by heat or by UV irradiation. An example of UV-activated initiator (i.e. photoinitiator) is 1-[4-(2-Hydroxyethoxy)-phenyl]-2-hydroxy-2-methyl-1-propane-1-one (available from Ciba Specialty Chemicals, Basel, Switzerland, under the trade name Irgacure(R) 2959).

Alternatively, a thermally initiated polymerization can be carried out. A suitable thermal initiator, Le., a compound generating free radicals upon heating, can be used. Exemplary thermal initiators are persulfates, azo initiator such as azobisisobutyronitrile (AIBN), peroxides such as benzoyl peroxide, and the like.

The following procedure describes a general method for coating a porous solid support with a fluorocarbon composition utilizing a photoinitiator. The substrate is wetted in methanol, rinsed in water, and soaked in the reactant solution comprising polymerizable fluorocarbon composition, MBAm cross-linkers and free radical photoinitiator for several minutes to ensure complete exchange. If the reactant solution is capable of wetting the substrate directly, the prewet-exchange steps are not necessary. The photoinitiators used to initiate polymerization of reactant solution on the surfaces of the substrate include for example, those described in J.-P. Fouassier, Photoinitiation, Photopolymerization, and Photocuring: Carl Hanser Verlag, Munich, Germany, 1995, pp. 20-93, and in Table 3-1, p. 21.

Polymerization of the reactant solution is effected by exposing the porous substrate saturated with polymerizable fluorocarbon composition/cross-linker/photoinitiator solution to ultra-violet, visible, or infrared radiation, to the dose sufficient to effect decomposition of the photoinitiator and generation of free radicals. A suitable source of ultra-violet radiation may include UV conveyor with two UV light sources, one on top and one on the bottom, as manufactured by Fusion UV Systems, Inc. (Gaithersburg, Md.). After the desired dose of radiation has been delivered by light source, the treated microporous substrate is rinsed in water and/or methanol to remove unreacted and oligomeric materials. The substrate is then dried and tested for rewet, flow and other properties.

If the fluorocarbon composition used is of the sulfonyl or carbonyl fluoride type, an additional hydrolysis step is required to convert the surface to the ion exchange form.

In another embodiment, surface modification of a porous solid support is carried out by grafting a fluorocarbon composition on the interior pore surfaces as well as the exterior, geometric surfaces.

In one example, a reactant solution preferably comprises polymerizable fluorocarbon composition with no crosslinker. In such an instance, an ionizing source of radiation such as, for example, an electron beam radiation source, is utilized to deliver an appropriate dose resulting in the generation of free radicals on the surface of the substrate on which fluorocarbon monomers can be grafted. One skilled in the art will be able to select a suitable radiation dose, where free radicals are generated on the surface of the porous substrate and fluorocarbon monomers polymerize and graft on to the surface without decomposition, using knowledge in the art coupled with routine experimentation.

In another example, a charged fluorocarbon composition can be obtained by coupling charged molecules directly to reactive groups introduced onto chemically inert fluorocarbon solid support. See, e.g., U.S. Pat. No. 4,642,285, which discloses a method of covalently attaching proteins, such as an antibody, onto an insoluble material. The insoluble material disclosed in the aforementioned patent is a commercially available material known as PROTAPOL DI/1 from Imperial Chemical Industries of Australia and New Zealand (ICIANZ). The material is available in a disc form and comprises a polytetrafluoroethylene (PTFE) backbone having isothiocyanopolystyrene groups grafted uniformly over its surface. Nucleophile terminated charged molecules (such as amines, thiols and the like) react with the isothiocyano groups resulting in covalent conjugation. See, e.g., Hermanson, Mallia and Smith, Immobilized Affinity Ligand Techniques, Academic Press, 1992.

In an alternative embodiment, surface modification of a porous solid support using a fluorocarbon composition is carried out by utilizing the layer by layer approach. Recently, preparation of thin films of polyelectrolyte complexes has been described using polyelectrolytes which are alternately deposited on a substrate. See Decher and Schlenoff, Eds., Multilayer Thin Films-Sequential Assembly of Nanocomposite Materials, Wiley-VCH, Weinheim (2003); Decher, Science, 277, 1232 (1997). Further, U.S. Pat. No. 5,208,111 describes a method for a buildup of multilayers by alternating dipping, i.e., cycling a substrate between two reservoirs containing aqueous solutions of polyelectrolytes of opposite charge, with an optional rinse step in polymer-free solution following each immersion. Each cycle adds a layer of polymer via ion pairing forces to the oppositely-charged surface and reverses the surface charge thereby priming the film for the addition of the next layer. Films prepared in this manner tend to be uniform, and cover the interior pore surfaces as well as the exterior, geometric surfaces of porous solid supports. Following this method, a negatively charged surface may be converted into a charged fluorocarbon composition by adsorbing a positively charged fluorocarbon polymer onto the surface.

Alternatively, a positively charged surface can be converted into a charged fluorocarbon composition by adsorbing a negatively charged fluorocarbon polymer onto the surface. See, e.g., U.S. Patent Publication No. 20100173224 A1, which provides examples of positively charged and negatively charged fluorinated polymers and copolymers that maybe used in the methods and compositions described herein.

### V. METHODS OF USING A SOLID SUPPORT HAVING A SURFACE MODIFIED WITH A CHARGED FLUOROCARBON COMPOSITION

Also described herein are methods of using fluorocarbon compositions according to the present invention. In some embodiments, a solid supports having a surface modified with a charged fluorocarbon composition can be used for selective removal of soluble impurities from aqueous solutions comprising a molecule of interest and one or more soluble impurities.

In some embodiments, a solid support having a surface modified with a charged fluorocarbon composition is incorporated into a suitable device, which can be used in a purification process.

In one embodiment, a solid support having a surface modified with a charged fluorocarbon composition is a microporous membrane encapsulated in a multi-layer device having an inlet and an outlet. In another embodiment, a solid support having a surface modified with a charged fluorocarbon composition is a porous chromatography resin packed into a column. In yet another embodiment, a solid support having a surface modified with a charged fluorocarbon composition is a porous monolith encapsulated in a suitable device providing flow inlet and outlet.

The following procedure describes a general method of using a device containing a solid support having a surface modified with a charged fluorocarbon composition, as described herein.

The solid support is fully wetted with water or a suitable aqueous buffer solution. A certain volume of a suitable aqueous buffer is subsequently flowed (or flushed) through the solid support to reduce the level of extractable compounds. The flush volume is usually between 1 and 1,000 volumes of the packed solid support, or between 5 to 100 volumes of the packed solid support. The solid support is then equilibrated with the aqueous buffer solution, which is substantially similar to the buffer solution containing molecule of interest and the impurities to be removed. Subsequently, the solution comprising the molecule of interest and the impurities is flowed through the device containing the solid support in manner to ensure contact between the solution components and the solid support, and the effluent is collected and analyzed for the concentration of molecule of interest and the impurities. The loading, i.e. the amount of the molecule of interest in the starting solution with respect to the volume of the solid support that the solution is flowed through, is chosen appropriately to achieve both effective impurity removal (highest LRV as defined above) and the highest yield of the molecule of interest. The acceptable range of loadings, usually expressed as weight of molecule of interest per volume of solid support, is determined prior to the preparative separation in a dedicated set of experiments. The range of acceptable loading can be between 0.01 to 10 kg/L, or between 0.5 and 5 kg/L. A greater loading is usually more desirable due to a more economical use of the solid support, lower loss of molecule of interest due to non-specific binding to the surfaces as well as remaining amount in the dead volume of devices and plumbing, and reducing the concentration of potential extractables from the solid support in the effluent.

The devices containing solid supports having a surface modified with a charged fluorocarbon composition CFC described herein (CFC devices) can be placed anywhere in a protein purification process, e.g., in an antibody purification process. Table 1 depicts examples of protein purification processes that incorporate CFC devices as one or more intermediate steps, which is shown by underline. It is understood that many variations of these processes may be used. The various steps that appear in Table 1 are described below.

“Protein capture” or “antibody capture,” step, as described herein, refers to the step in a protein purification process which involves isolating the protein of interest from the clarified or unclarified cell culture fluid sample by performing at least the following two steps: i) subjecting the cell culture fluid to a step selected from one or more of: adsorption of the protein of interest on a chromatography resin, a membrane, a monolith, a woven or non-woven media; precipitation, flocculation, crystallization, binding to a soluble small molecule or a polymeric ligand, thereby to obtain a protein phase comprising the protein of interest such as, e.g., an antibody; and (ii) reconstituting the protein of interest by eluting or dissolution of the protein into a suitable buffer solution.

Bind/elute purification is an optional process step consisting of binding the protein of interest to a suitable chromatography media, optionally washing the bound protein, and eluting it with appropriate buffer solution.

Flow-through AEX polishing is an optional process step consisting of flowing the solution of protein of interest through a suitable AEX chromatography media without significantly binding of the protein of interest to the media.

Activated Carbon Flow-through is an optional purification step designed to remove various process-related impurities, as described in co-pending provisional patent application No. 61/575,349, incorporated by reference herein.

Flow-through aggregate removal is an optional purification step designed to remove various aggregated species of target proteins, as described in co-pending provisional application No. 61/609,533, incorporated by reference herein.

Virus filtration consists of flowing the protein solution through a porous membrane, which can be in the form of flat sheet or hollow fiber that retains the viral particles to high degree of LRV, while passing substantially all protein of interest.

TABLE 1

| | Step 1 | Step 2 | Step 3 | Step 4 | Step 5 | Step 6 |
|---|---|---|---|---|---|---|
| Process | Antibody | Bind/elute | Flow-through | Flow- | CFC | Virus |
| A | capture | Purification | AEX | through | device | Filtration |
| | | | polishing | aggregate | | |
| | | | | removal | | |
| Process | Antibody | Bind/elute | Flow-through | CFC device | Virus | |
| B | capture | Purification | AEX | | Filtration | |
| | | | polishing | | | |
| Process | Antibody | Flow- | Bind/elute | Flow- | CFC | Virus |
| C | capture | through | Purification | through | device | Filtration |
| | | aggregate | | AEX | | |
| | | removal | | polishing | | |
| Process | Flow- | Antibody | Bind/elute | Flow- | CFC | Virus |
| D | through | capture | Purification | through | device | Filtration |
| | aggregate | | | AEX | | |
| | removal | | | polishing | | |
| Process | Antibody | Flow- | Flow-through | CFC device | Virus | |
| E | capture | through | AEX | | Filtration | |
| | | aggregate | polishing | | | |
| | | removal | | | | |
| Process | Antibody | Flow- | CFC device | Virus | | |
| F | capture | through | | Filtration | | |
| | | AEX | | | | |
| | | polishing | | | | |
| Process | Antibody | Flow- | CFC device | Bind/elute | Virus | |
| G | capture | through | | Purification | Filtration | |
| | | AEX | | | | |
| | | polishing | | | | |
| Process | Antibody | Activated | Flow-through | CFC device | Virus | |
| H | capture | Carbon | AEX | | Filtration | |
| | | Flow- | polishing | | | |
| | | through | | | | |
| Process | Antibody | Activated | Flow-through | Flow- | CFC | Virus |
| I | capture | Carbon | AEX | through | device | Filtration |
| | | Flow- | polishing | aggregate | | |
| | | through | | removal | | |

It is understood that in the Table 1 above, the step of Antibody Capture, as well as Bind/Elute Purification, can be operated in any of three modes: (1) batch mode, where the capture media is loaded with target protein, loading is stopped, media is washed and eluted, and the pool is collected; (2) semi-continuous mode, where typically, the loading of sample is performed continuously, i.e. without stopping the fluid flow, whereas the elution is performed intermittently; (3) full continuous mode, wherein both loading and elution are performed continuously.

This invention is further illustrated by the following examples which should not be construed as limiting. The contents of all references, patents and published patent applications cited throughout this application, as well as the Figures, are incorporated herein by reference.

### EXAMPLES

### Example 1

### Preparation of a Microporous Membrane Having a Surface Modified with a Fluorocarbon Composition

In a representative experiment, 5% aqueous dispersions of charged fluorocarbon compositions (CFC) from Table 2 are prepared by dilution or exchanging alcohol for water. The 5% dispersion is neutralized to pH 7 using a pH meter and 2M aqueous lithium hydroxide. Hydrophilic microporous membrane with a pore size rating of 0.65 micron made from a ultra-high molecular weight polyethylene (UHMWPE, or UPE) is used throughout the experiments described herein, as a porous substrate, and referred to herein as the “unmodified membrane.” The unmodified membrane is cut into 5.25×5.25 inch squares and put into a 6×8 inch polyethylene bag. A 3 mL aliquot of the dispersion is pipetted onto the center of the membrane in the bag. A hand ink roller is used to carefully spread the dispersion and displace the air out of the pores. The roller is then used to firmly push out the excess liquid. The bag is cut open and the membrane is blow dried at room temperature and then dried at 90° C. for 15 minutes. The membrane is cooled to room temperature and carefully wetted with Milli-Q water and rinsed 3 times with fresh Milli-Q water. The CFC-coated membrane is then dried at room temperature. Prior to static protein binding experiments described herein, the membranes are wet with Milli-Q water and then put into 1 mL of corresponding buffer solution.

The membranes modified with CFC from aqueous dispersion remain wettable with water and have a Critical Wetting Surface Tension (CWST) of >72 dynes/cm. Typical pure water flux for the modified membrane is reduced from 2,000 LMH/psi for unmodified membrane to about 500-800 LMH/psi. Table 2 shows the typical CFC add-on percentage for each equivalent weight.

“Micro” devices are manufactured using pre-molded polypropylene parts. The coated membrane is punched into 25 mm disks. 4 layers of the 125 micron CFC-modified membrane are put into each device and overmolded. The active filtration diameter is about 20 mm, which for 4 layers corresponds to a volume of 0.157 mL per device. Efficient flow distribution inside this device configuration is verified by passing a dilute solution of a positively charged dye, Methylene Blue, and subsequently opening the devices for visual inspection.

TABLE 2
Commercially available charged fluorocarbon compositions

| | | | Equlvalent | |
|---|---|---|---|---|
| | | | weight | |
| | | | (g/mole | |
| Manufacturer | Name | Code | —SO₃H) | Description |
| DuPont | Nafion ® | D1020 | 1000 | 10% in water |
| | Nafion ® | D1021 | 1100 | 10% in water |
| | Nafion ® | D520 | 1000 | 5% in alcohol |
| | | | | and water |
| | Nafion ® | D521 | 1100 | 5% in alcohol |
| | | | | and water |
| | Nafion ® | D2020 | 1000 | 20% in alcohol |
| | | | | and water |
| | Nafion ® | D2021 | 1100 | 20% in alcohol |
| | | | | and water |
| Solvay Solexis | Aquivion ™ | D83-20B | 830 | 20% in water |
| | Aquivion ™ | D83-06A | 830 | 6% in alcohol |
| | | | | and water |

TABLE 3
Typical weight add-on of charged fluorocarbon composition

| | Equlvalent Weight | Typical CFC Add on (w/w %) |
|---|---|---|
| Manufacturer | (g/mole —SO₃H) | from 5% aqueous dispersions |
| DuPont | 1000 | 13-18 |
| DuPont | 1100 | 15-20 |
| Solvay Solexis | 830 | 10-13 |

### Example 2

### CFC-Modified Membrane Binds Isozyme at Both Low as Well as High Salt Concentration

This Example illustrates that CFC-modified membrane can bind a target molecule, e.g., Lysozyme in this case, at both low salt concentration (25 mM buffer, no added salt) as well as high salt concentration (25 mM buffer with 0.5M sodium chloride).

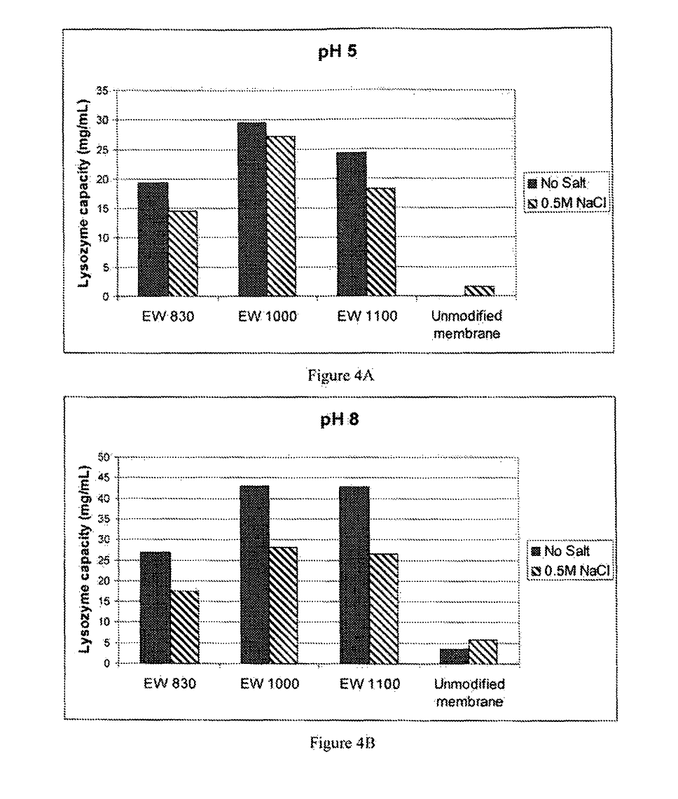

Static capacity measurements are performed using 25 mm disks of membrane prepared in Example 1. Disks are first soaked in corresponding buffer solution and then submerged in I g/L of Lysozyme for 16 hours. Residual protein concentrations are measured using UV absorption at 280 nm. As depicted in FIG. 4, it is demonstrated that the binding capacity is only slightly reduced at high salt concentration, especially at pH 5, indicating very strong salt tolerance by CFC-modified membranes

### Example 3

### CFC-Modified Membranes can Bind BSA at Both Low Salt Concentration as Well as High Salt Concentration

In another experiment, it was demonstrated that the CFC-modified membranes can bind another target molecule, e.g., Bovine Serum Albumin (BSA), at both low salt concentration (25 mM buffer, no salt added) as well as high salt concentration (25 mM buffer with 0.5M sodium chloride).

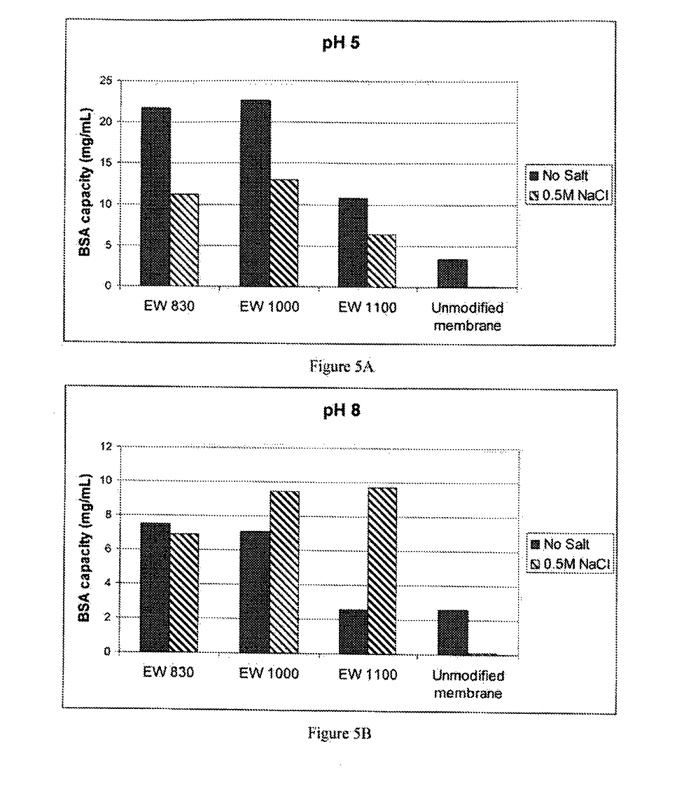

Static capacity measurements are performed using 25 mm disks of membrane prepared in Example 1. Disks are first soaked in corresponding buffer solution and then submerged in 1 g/L of BSA for 16 hours. Residual protein concentrations are measured using UV absorption at 280 nm. As demonstrated in FIG. 5, at pH 5, while some drop in capacity upon salt addition is observed, but significant capacity is still retained. At pH 8, on the contrary, the binding capacity increases as salt is added.

This surprising phenomenon demonstrates the unique and unexpected properties of the charged fluorocarbon compositions described herein, i.e., maintaining high binding capacity at both low and high salt concentrations, at both extremes of the practical operating pH range for biological molecules, and for proteins of both low isoelectric point (BSA) and high isoelectric point (Lysozyme).

### Example 4

### CFC-Modified Membrane Exhibits Static Binding Capacity for Human Polyclonal IgG

This representative experiment illustrates that CFC-modified membrane exhibits static binding capacity for human polyclonal IgG

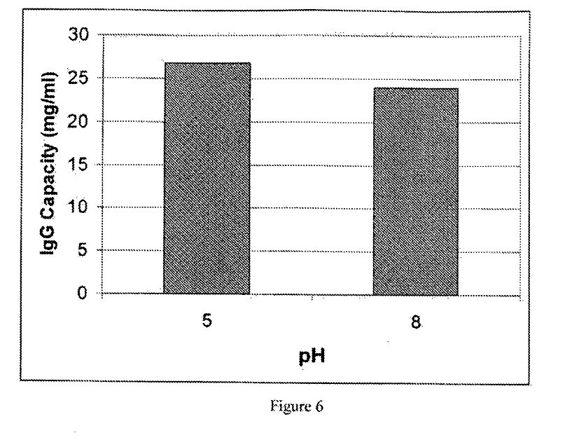

The procedure in Example 3 was followed using 1 g/L solution of polyclonal human IgG from SeraCare Life Sciences, Inc., Milford, Mass. The data in FIG. 6 indicate very little effect of salt on IgG binding capacity at pH 5 as compared to pH 8, both performed at 0.5M NaCl.

### Example 5

### CFC-Modified Membrane Exhibits Static Binding Capacity for Host Cell Proteins

This representative experiment illustrates that CFC-modified membrane exhibits static binding capacity for host cell protein. Host cell protein (pI 3-10), or HCP, is a mixture of more than 100 different proteins that are produced by host cells (e.g., CHO cells) and are usually the main contaminant that appears with a protein of interest (e.g., a therapeutic antibody).

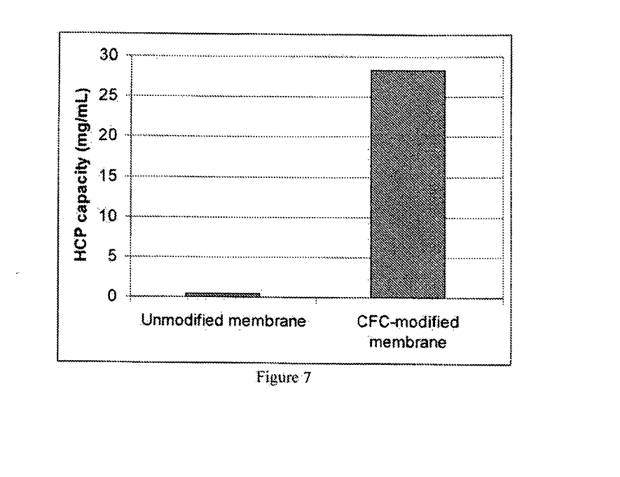

In one experiment, HCP mixture was produced from a null mammalian cell culture CHO—S. It was clarified using centrifugation followed by sterile filtration, and further purified using a cation-exchange bind/elute chromatographic step. FIG. 7 demonstrates that a CFC-modified membrane binds a significant amount of HCP compared to unmodified membrane.

### Example 6

### CFC-Modified Membrane Exhibits Selective Removal of HCP from Clarified Cell Culture Fluid

This representative experiment demonstrates that CFC-modified membrane can be used for selective removal of host cell protein from a clarified cell culture fluid.

In one experiment, the starting cell culture is a CHO—S cell line producing a monoclonal antibody, referred to as Mab04. The culture is clarified prior to loading using centrifugation followed by sterile filtration. A total of approximately 20 mL (125 of membrane column volumes, or CVs) of the clarified cell culture is loaded onto the CFC-modified membrane media, the effluent is collected and analyzed for mAb, HCP and DNA concentrations. The results are shown in Table 4.

As demonstrated by this experiment, the CFC-modified membrane exhibits selectivity for both Host Cell Protein and DNA.

TABLE 4

| Measurement | Feed | Effluent |
|---|---|---|
| IgG (g/L) | 0.43 | 0.48* |
| HCP (ppm) | 481658 | 170511 |
| DNA (ug/mL) | 9.94 | 3.85 |
| IgG yield (%) | | 110.7* |
| HCP LRV | | 0.45 |
| DNA LRV | | 0.46 |

*Inherent errors of IgG concentration measurements in this range cause the yield to appear above theoretically feasible 100%.

### Example 7

### CFC-Modified Membrane can be Used for Removal of HCP from a Protein A Elution Pool

In a representative experiment, it is demonstrated that CFC-modified membranes can be used for the removal of host cell proteins from a protein A elution pool.

A similar experiment to Example 6 is performed using a Protein A chromatography elution pool as the feed material. This feed is at pH 5 in sodium acetate buffer with a MAb04 concentration of approximately 7 g/L. The impurity levels (HCP and DNA) of this feed are expected to be significantly lower than in the clarified feed of Example 6. The media is loaded with 32 mL (200 CVs) of the protein A elution. The results are shown in Table 5, which demonstrate that the CFC-modified membrane exhibits selectivity for both HCP and DNA using a protein A elution pool.

TABLE 5

| Measurement | Feed | Effluent |
|---|---|---|
| IgG (g/L) | 7.0 | 6.5 |
| HCP (ppm) | 596 | 300 |
| DNA (ug/mL) | 2.89 | 0.13 |
| IgG yield (%) | | 93.3 |
| HCP LRV | | 0.30 |
| DNA LRV | | 1.31 |

### Example 8

### CFC-Modified Membrane can be Used for Removal of Host Cell Proteins from a Protein A Elution Pool that has been Further Purified Usine AEX-Flow Through Step

In a representative experiment, it was demonstrated that CFC-modified membrane can be used for the removal of host cell protein from a Protein A elution pool that has been further purified using a AEX flow-through step.

The same protein A elution as in Example 7 is first purified using an anion-exchange membrane adsorber, ChromaSorb™ available from EMD Millipore Corp. ChromaSorb device is loaded to 2.5 kg/L (375 CVs) at pH 8, according to the manufacturer's instructions. Following the ChromaSorb step, the flow-through pool is processed for loading onto the CFC-modified membrane. The pH of the pool is lowered to pH 5 using glacial acetic acid. Another 15 mL of 50 mM sodium acetate pH 5 is added to the approximately 30 mL of ChromaSorb™ flow-through solution in order to lower the conductivity. The final pH is 5.07 with a conductivity of 3.7 mS/cm. Approximately 40 mL of this material is loaded onto the CFC-modified membrane at 1.6 mL/min. The results of this experiment are presented in Table 6, which demonstrate that the CFC-modified membrane has selectivity for HCP.

TABLE 6

| | Feed | Effluent |
|---|---|---|
| IgG (g/L) | 4.17 | 3.7 |
| HCP (ppm) | 172 | 14.8 |
| DNA (ug/mL) | Not detected | Not detected |
| IgG yield (%) | | 89 |
| HCP LRV | | 1.06 |
| DNA LRV | | N/A |

### Example 9

### CFC-Modified Membranes Remove HCP from an Antibody Feed at Both Low as Well as Blab Salt Concentrations

In this representative experiment, it is demonstrated that the CFC-modified membranes can remove HCP from an antibody feed at both low as well as high salt concentrations.

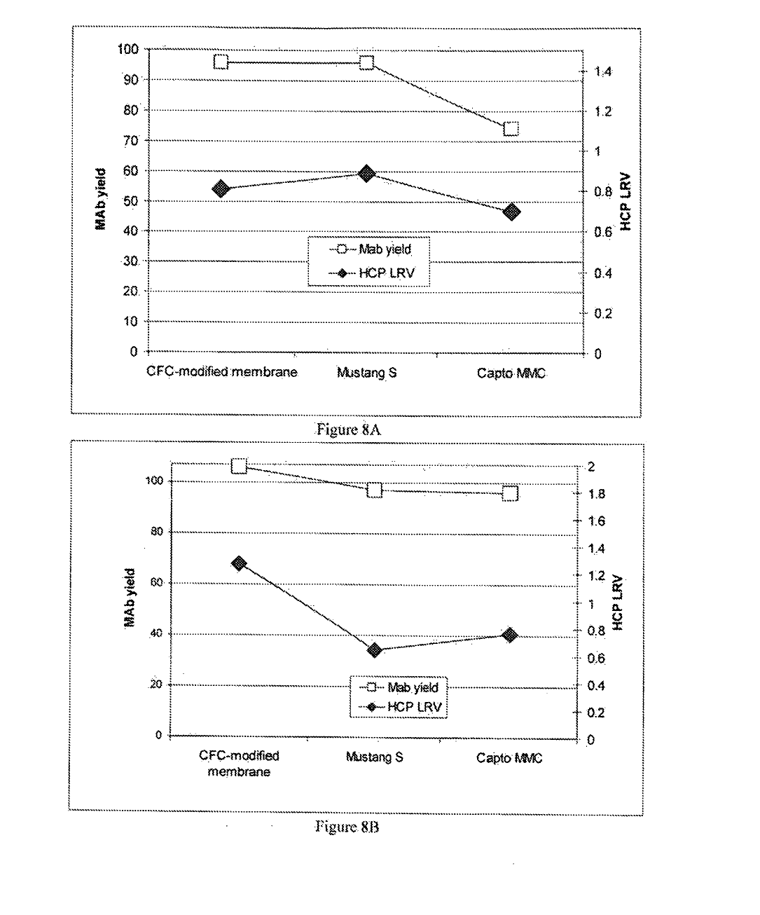

A similar protein A elution as in Example 7 is first purified using an anion-exchange membrane adsorber, ChromaSorb™ available from EMD Millipore Corp. ChromaSorb™ device is loaded to 2 kg/L (320 CVs) at pH 7, according to the manufacturer's instructions. The effluent pool was collected and found to contain 175.4 ppm of HCP. Following the ChromaSorb™ step, the pH of the pool is lowered to pH 5 using glacial acetic acid, and the flow-through pool is loaded onto the CFC-modified membrane, as well as commercially available benchmarks: Pall Mustang® S (available from Thermo Scientific, Waltham, Mass.), and Capto™ MMC (GE Healtcare) HiTrap™ column. The CFC-modified membrane and Mustang® S were loaded to 200 CV's, and Capto™ MMC was loaded to 150 CV's. The effluents were collected and assayed for MAb04 yield and HCP concentration. The results are shown in FIG. 8A.

In another experiment, a similar protein A elution as in Example 7 is first purified using an anion-exchange membrane adsorber, ChromaSorb™ available from EMD Millipore Corp. ChromaSorb™ device is loaded to 3.1 kg/L (320 CVs) at pH 7, according to the manufacturer's instructions. The effluent pool was collected and found to contain 347 ppm of HCP. Following the ChromaSorb™ step, the pH of the pool is lowered to pH 5 using glacial acetic acid and the conductivity is increased by adding sodium chloride to a final concentration of 250 mM. The flow-through pool is loaded onto the CFC-modified membrane, as well as commercially available benchmarks: Pall Mustang® S (available from Thermo Scientific, Waltham, Mass.), and Capto™ MMC (GE Healtcare) HiTrap™ column. The CFC-modified membrane and Mustang® S were loaded to 200 CV's, and Captom MMC was loaded to 150 CV's. The effluents were collected and assayed for MAb04 yield and HCP concentration. The results are shown in FIG. 8B.

It is observed that a CFC-modified membrane successfully removes a significant portion of HCP. It is also observed that while the performance at low salt concentration is comparable to a commercially available cation-exchange membrane, Pall Mustang® S, it does not deteriorate at high salt concentration (in fact, HCP LRV is slightly higher), thereby demonstrating that the CFC-modified membrane is markedly superior to the cation-exchange commercially available membrane, used as a benchmark herein.

### Example 10

### CFC-Modified Membrane can be Used for Removal of Host Cell Protein from a Purified Antibody Feed

In a representative experiment, a harvested cell culture fluid from Chinese Hamster Ovary (CHO) cells expressing a monoclonal antibody, is clarified and purified according to a procedure disclosed in the published U.S. Patent Publication No. 20090232737. The solution buffer is exchanged into 25 mM Tris-HCl, pH 8.0, and the antibody concentration is adjusted to 2.0 g/L by addition of pure antibody. The solution is first purified by passing it through a EMD Millipore ChromaSorb™ membrane adsorber device for a total loading of 40 g/L, the pH is lowered to 5.0, and the effluent is further purified by passing through a Micro device containing CFC-modified membrane.

As depicted in Table 7, the CFC-modified membrane exhibits selectivity for HCP.

TABLE 7

| Measurement | Feed | Effluent |
|---|---|---|
| IgG (g/L) | 2.4 | 2.38 |
| HCP (ppm) | 429 | 121.8 |
| DNA (ug/mL) | Not detected | Not detected |
| IgG yield (%) | | 99 |
| HCP LRV | | 0.55 |
| DNA LRV | | N/A |

### Example 12

### CFC-Modified Membrane can be Used for Salt-Tolerant Virus Removal

In a representative experiment, it is demonstrated that the CFC-modified membrane can be used for salt-tolerant virus removal.

CFC-modified membrane from Example 1 is encapsulated into a 0.16 mL “Micro” device described above. The devices are wetted with water. Protein A elution of Mab04 at 10 g/L is spiked with XMuLV virus and adjusted to the necessary pH and conductivity using 1 M Tris base and 5.5 M NaCl stock solutions. In the case of pH 7.0 and 100 mM NaCl, a precipitation of antibody was observed, so the virus quantification for that condition was not performed. The devices are loaded to I kg/L of antibody, and the pools are assayed for residual XMuLV. Surprisingly, a strong retention of virus is observed at both low salt concentration and at 100 mM NaCl.

TABLE 8
LRV of XMuLV.

| | [NaCl] | | | |
|---|---|---|---|---|
| pH | 0 | 20 | 50 | 100 |
| 5 | n/m | >4.02 | n/m | 3.39 |
| 7 | >3.77 | >4.02 | >4.15 | n/m |

n/m—not measured

The specification is most thoroughly understood in light of the teachings of the references cited within the specification which are hereby incorporated by reference. The embodiments within the specification provide an illustration of embodiments in this invention and should not be construed to limit its scope. The skilled artisan readily recognizes that many other embodiments are encompassed by this invention. All publications and inventions are incorporated by reference in their entirety. To the extent that the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

What is claimed is:

1. A method of removing a target biomolecule from a sample, the method comprising:
(i) providing a sample comprising the target biomolecule; and
(ii) contacting the sample with a porous solid support having a surface modified with a charged fluorocarbon composition, wherein the target biomolecule binds to the modified solid support,
thereby removing the target biomolecule from the sample.

2. The method of claim 1, wherein the target biomolecule is a soluble impurity.

3. The method of claim 2, wherein the soluble impurity is a host cell protein.

4. The method of claim 1, wherein the target biomolecule is a virus or viral particle.

5. The method of claim 4, further comprising the step of recovering the bound target biomolecule.

6. A method of reducing the level of one or more impurities in a sample comprising a target protein and the one or more impurities, the method comprising the steps of:
(i) providing a sample comprising a target protein and one or more impurities;
(ii) contacting the sample with a porous solid support having a surface modified with a charged fluorocarbon composition; and
(iii) obtaining an effluent,
wherein the effluent comprises a lower level of the one or more impurities relative to the level of the one or more impurities in the sample in (i).

7. The method of claim 6, wherein the sample comprises a cell culture feed.

8. The method of claim 1, the sample comprises a salt concentration of at least 100 mM.

9. The method of claim 6, wherein the sample comprises a salt concentration of at least 100 mM.

10. The method of claim 1, wherein the charged fluorocarbon composition comprises the structure: 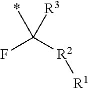
wherein R₁ is selected from a group consisting of a sulfonic, a sulfate, a phosphonic, a phosphoric and a carboxylic residue; R₂ is an optional linking unit that is selected from the group consisting of short saturated and unsaturated hydrocarbon groups, R₃ is a F, Cl or a C₁ to C₁₀ perfluoroalkyl radical and F is a fluorine group.

11. The method of claim 6, wherein the charged fluorocarbon composition comprises the structure: 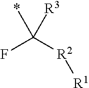
wherein R₁ is selected from a group consisting of a sulfonic, a sulfate, a phosphonic, a phosphoric and a carboxylic residue; R₂ is an optional linking unit that is selected from the group consisting of short saturated and unsaturated hydrocarbon groups, R₃ is a F, Cl or a C₁ to C₁₀ perfluoroalkyl radical and F is a fluorine group.

12. The method of claim 10, wherein R2 is a CₓH_{y} group, wherein x ranges from 1 to about 10 and y ranges from 0 to about 20 of an alkoxy group, an ester, an amide and the like.

13. The method of claim 11, wherein R2 is a CₓH_{y} group, wherein x ranges from 1 to about 10 and y ranges from 0 to about 20 of an alkoxy group, an ester, an amide and the like.

14. A flow-through process for purifying a target molecule from a sample comprising the steps of:
(a) subjecting a sample comprising the target molecule and one or more impurities to a Protein A affinity chromatography process;
(b) flowing a Protein A eluate from step (a) through an activated carbon media;
(c) flowing the output from (b) through an anion exchange chromatography media;
(d) flowing the output from (c) through a cation exchange chromatography media;
(e) flowing the output from (d) through a porous solid support having a surface modified with a charged fluorocarbon composition;
(f) flowing the output from (e) through a virus filtration media; and
(g) recovering the output from (f), thereby to purify the target molecule.

15. The method of claim 14, wherein the target molecule is an antibody.

16. The method of claim 15, wherein the antibody is a monoclonal antibody.
